SEQUENCE LISTING (43 sequences)

SEQ ID NO: 1 - protein, 225 aa, Escherichia coli
MIMDLASLRAQQIELASSVIREDRLDKDPPDLIAGADVGFEQGGEVTRAAMVLLKYPSLELVEYKVARIATTMPYIPGFLSFREYPALLAAWEMLSQKPDLVFVDGHGISHPRRLGVASHFGLLVDVPTIGVAKKRLCGKFEPLSSEPGALAPLMDKGEQLAWVWRSKARCNPLFIATGHRVSVDSALAWVQRCMKGYRLPEPTRWADAVASERPAFVRYTANQP

SEQ ID NO: 2 - protein, 225 aa, Artificial Sequence
  Mutant E. coli Endonuclease V
MIMDLASLRAQQIELASSVIREDRLDKDPPDLIAGADVGFEQGGEVTRAAMVLLKYPSLELVEYKVARIATTMPAIPGFLSFREYPALLAAWEMLSQKPDLVFVDGHGISHPRRLGVASHFGLLVDVPTIGVAKKRLCGKFEPLSSEPGALAPLMDKGEQLAWVWRSKARCNPLFIATGHRVSVDSALAWVQRCMKGYRLPEPTRWADAVASERPAFVRYTANQP

SEQ ID NO: 3 - protein, 221 aa, Archaeoglobus fulgidus
MLQMNLEELRRIQEEMSRSVVLEDLIPLEELEYVVGVDQAFISDEVVSCAVKLTFPELEVVDKAVRVEKVTFPYIPTFLMFREGEPAVNAVKGLVDDRAAIMVDGSGIAHPRRCGLATYIALKLRKPTVGITKKRLFGEMVEVEDGLWRLLDGSETIGYALKSCRRCKPIFISPGSYISPDSALELTRKCLKGYKLPEPIRIADKLTKEVKRELTPTSKLK

SEQ ID NO: 4 - protein, 221 aa, Artificial Sequence
  Mutant Afu Endonuclease V
MLQMNLEELRRIQEEMSRSVVLEDLIPLEELEYVVGVDQAFISDEVVSCAVKLTFPELEVVDKAVRVEKVTFPAIPTFLMFREGEPAVNAVKGLVDDRAAIMVDGSGIAHPRRCGLATYIALKLRKPTVGITKKRLFGEMVEVEDGLWRLLDGSETIGYALKSCRRCKPIFISPGSYISPDSALELTRKCLKGYKLPEPIRIADKLTKEVKRELTPTSKLK

SEQ ID NO: 5 - protein, 225 aa, Termotoga maritima
MDYRQLHRWDLPPEEAIKVQNELRKKIKLTPYEGEPEYVAGVDLSFPGKEEGLAVIVVLEYPSFKILEVVSERGEITFPYIPGLLAFREGPLFLKAWEKLRTKPDVVVFDGQGLAHPRKLGIASHMGLFIEIPTIGVAKSRLYGTFKMPEDKRCSWSYLYDGEEIIGCVIRTKEGSAPIFVSPGHLMDVESSKRLIKAFTLPGRRIPEPTRLAHIYTQRLKKGLF

SEQ ID NO: 6 - protein, 225 aa, Artificial Sequence
  Mutant Tma Endonuclease V
MDYRQLHRWDLPPEEAIKVQNELRKKIKLTPYEGEPEYVAGVDLSFPGKEEGLAVIVVLEYPSFKILEVVSERGEITFPAIPGLLAFREGPLFLKAWEKLRTKPDVVVFDGQGLAHPRKLGIASHMGLFIEIPTIGVAKSRLYGTFKMPEDKRCSWSYLYDGEEIIGCVIRTKEGSAPIFVSPGHLMDVESSKRLIKAFTLPGRRIPEPTRLAHIYTQRLKKGLF

SEQ ID NO: 7 - DNA, 18 bp, Artificial Sequence
  Synthetic Primer Sequence
acagccgctagtcctant

SEQ ID NO: 8 - DNA, 17 bp, Artificial Sequence
  Synthetic Primer Sequence
cccgcaaagttcctcna

SEQ ID NO: 9 - DNA, 16 bp, Artificial Sequence
  Synthetic Primer Sequence
accgggtctccttcnc

SEQ ID NO: 10 - DNA, 18 bp, Artificial Sequence
  Synthetic Primer Sequence
gctgcgcgaaccacttna

SEQ ID NO: 11 - DNA, 15 bp, Artificial Sequence
  Synthetic Primer Sequence
ccgtacccggagcnc

SEQ ID NO: 12 - DNA, 17 bp, Artificial Sequence
  Synthetic Primer Sequence
cagattcccgccaganc

SEQ ID NO: 13 - DNA, 15 bp, Artificial Sequence
  Synthetic Primer Sequence
ggcgaaccgatcanc

SEQ ID NO: 14 - DNA, 14 bp, Artificial Sequence
  Synthetic Primer Sequence
cggcggattcgcnc

SEQ ID NO: 15 - DNA, 17 bp, Artificial Sequence
  Synthetic Primer Sequence
ggttgacatcaccccnc

SEQ ID NO: 16 - DNA, 17 bp, Artificial Sequence
  Synthetic Primer Sequence
gagcacctcttccagnc

SEQ ID NO: 17 - DNA, 18 bp, Artificial Sequence
  Synthetic Primer Sequence
cgatcggagacagctcnt

SEQ ID NO: 18 - DNA, 18 bp, Artificial Sequence
  Synthetic Primer Sequence
acagccgctagtcctant

SEQ ID NO: 19 - DNA, 17 bp, Artificial Sequence
  Synthetic Primer Sequence
cccgcaaagttcctcna

SEQ ID NO: 20 - DNA, 16 bp, Artificial Sequence
  Synthetic Primer Sequence
accgggtctccttcnc

SEQ ID NO: 21 - DNA, 18 bp, Artificial Sequence
  Synthetic Primer Sequence
gctgcgcgaaccacttna

SEQ ID NO: 22 - DNA, 15 bp, Artificial Sequence
  Synthetic Primer Sequence
ccgtacccggagcnc

SEQ ID NO: 23 - DNA, 17 bp, Artificial Sequence
  Synthetic Primer Sequence
cagattcccgccaganc

SEQ ID NO: 24 - DNA, 15 bp, Artificial Sequence
  Synthetic Primer Sequence
ggcgaaccgatcanc

SEQ ID NO: 25 - DNA, 14 bp, Artificial Sequence
  Synthetic Primer Sequence
cggcggattcgcnc

SEQ ID NO: 26 - DNA, 17 bp, Artificial Sequence
  Synthetic Primer Sequence
ggttgacatcaccccnc

SEQ ID NO: 27 - DNA, 17 bp, Artificial Sequence
  Synthetic Primer Sequence
gagcacctcttccagnc

SEQ ID NO: 28 - DNA, 18 bp, Artificial Sequence
  Synthetic Primer Sequence
cgatcggagacagctcnt

SEQ ID NO: 29 - DNA, 21 bp, Artificial Sequence
  Synthetic Primer Sequence
catgaagtgctggaaggatnc

SEQ ID NO: 30 - DNA, 21 bp, Artificial Sequence
  Synthetic Primer Sequence
tcctctaagggctctcgttng

SEQ ID NO: 31 - DNA, 21 bp, Artificial Sequence
  Synthetic Primer Sequence
aaattatcgcggcgaacggnc

SEQ ID NO: 32 - DNA, 19 bp, Artificial Sequence
  Synthetic Primer Sequence
ggcagattcccgccaganc

SEQ ID NO: 33 - DNA, 21 bp, Artificial Sequence
  Synthetic Primer Sequence
aaaacagccgctagtcctant

SEQ ID NO: 34 - DNA, 20 bp, Artificial Sequence
  Synthetic Primer Sequence
tcgcccgcaaagttcctcna

SEQ ID NO: 35 - DNA, 20 bp, Artificial Sequence
  Synthetic Primer Sequence
ccaaaccgggtctccttcnc

SEQ ID NO: 36 - DNA, 21 bp, Artificial Sequence
  Synthetic Primer Sequence
taagctgcgcgaaccacttna

SEQ ID NO: 37 - DNA, 20 bp, Artificial Sequence
  Synthetic Primer Sequence
ctgggttgacatcaccccnc

SEQ ID NO: 38 - DNA, 21 bp, Artificial Sequence
  Synthetic Primer Sequence
aagtcctcgatcggagacanc

SEQ ID NO: 39 - DNA, 20 bp, Artificial Sequence
  Synthetic Primer Sequence
gacaacgacatcgacccgna

SEQ ID NO: 40 - DNA, 21 bp, Artificial Sequence
  Synthetic Primer Sequence
cgtcgaaacgagggtcagana

SEQ ID NO: 41 - DNA, 20 bp, Artificial Sequence
  Synthetic Primer Sequence
ctcgtcgacgggtgccttna

SEQ ID NO: 42 - DNA, 23 bp, Artificial Sequence
  Synthetic Primer Sequence
gtacgtcatgtccttgtctttnc

SEQ ID NO: 43 - DNA, 22 bp, Artificial Sequence
  Synthetic Primer Sequence
tcaccggtgttgttgttgatna

(19) United States
(12) Patent Application Publication
(10) Pub. No.: US 2015/0167065 A1
(43) Pub. Date: Jun. 18, 2015

(54) ISOTHERMAL AMPLIFICATION OF NUCLEIC ACIDS WITHIN A POROUS MATRIX

(71) Applicant: General Electric Company, Schenectady, NY (US)

(72) Inventors: John Richard Nelson, Clifton Park, NY (US); David Roger Moore, Rexford, NY (US); Bing Li, Clifton Park, NY (US); Robert Scott Duthie, Schenectady, NY (US); Patrick McCoy Spooner, Slingerlands, NY (US)

(73) Assignee: General Electric Company, Schenectady, NY (US)

(21) Appl. No.: 14/106,264

(22) Filed: Dec. 13, 2013

(51) Int. Cl. C12Q 1/68

(52) U.S. Cl. CPC C12Q 1/6844

(57) ABSTRACT

Provided herein are methods for amplification a target dsDNA that is impregnated within a porous matrix using endonuclease-assisted DNA amplification. The amplicons may be subsequent detected within the porous matrix or may be eluted out of the porous matrix. Methods for extracting a genetic material from a biological sample using endonuclease-assisted DNA amplification within a porous matrix are also provided.

### FEDERALLY SPONSORED RESEARCH & DEVELOPMENT

This invention was made with Government support under contract number HR0011-11-2-0007, held by the University of Washington awarded by the Defense Advanced Research Projects Agency (DARPA). The Government has certain rights in the invention.

### FIELD OF INVENTION

The invention generally relates to isothermal amplification of a double stranded DNA (dsDNA) within a porous matrix. It further relates to amplification of a dsDNA (e.g., a genomic DNA) that is impregnated within a porous matrix using an endonuclease-assisted nucleic acid amplification and subsequent detection of amplicons within the porous matrix.

### BACKGROUND

With the development of a variety of techniques for isolation, amplification and detection of nucleic acids, nucleic acid-based assays have emerged over the years as powerful tools for various applications such as diagnostic and forensic analysis. However, even today, immunoassays have more widespread acceptance then nucleic acid-based assays due to their easy formats and lower operational costs Immunoassays are less complex than nucleic acid-based assays since they are simple detection assays and do not involve any target amplification step. In contrast, amplification of a nucleic acid target (e.g., DNA amplification) is a critical step in many of the nucleic acid-based assays. DNA amplification is a process of replicating a target DNA to generate multiple copies of it. Since individual strands of a dsDNA are antiparallel and complementary, each strand may serve as a template strand for the production of its complementary strand. The template strand is preserved as a whole or as a truncated portion and the complementary strand is assembled from deoxynucleoside triphosphates (dNTPs) by a DNA polymerase. The complementary strand synthesis proceeds in 5′→3′ direction starting from the 3′ terminal end of a primer sequence that is hybridized to the template strand.

For most of the currently known DNA amplification techniques, expensive and/or complex equipment and higher levels of skilled labor are required. For nucleic acid-based analysis to become widely used for clinical or industrial applications, complexity of assays/instrumentation and their cost need to be reduced. Specifically, if nucleic-acid based tests were to be performed at the point of care (POC) level (e.g., near patient or near process), simple, easy to use, cost-competitive systems are essential. DNA-based assays involving thermal cycling amplification have been performed in a lateral flow stick employing an associated thermally-regulatable apparatus. However, such thermal cycling amplification methods are less than ideal, for example, for POC applications. Even though isothermal nucleic acid amplification (e.g., SMART reaction) of a target DNA putatively immobilized in a porous matrix (e.g., FTA paper) has been attempted, such methods involved prior heating of the immobilized, target dsDNA to thermally denature the target dsDNA to its single stranded counterparts.

DNA detection techniques employing simplified DNA amplification methods that do not require target dsDNA denaturation prior to its amplification would offer several potential advantages for processing and screening of a broad range of sample types. Further, along with simpler and robust sample preparation and processing methods for trace and/or dilute target nucleic acids, such techniques would greatly facilitate DNA-based assays in POC and field-deployed assays.

### BRIEF DESCRIPTION

In some embodiments, a method for producing at least one amplicon based on a target dsDNA within a porous matrix is provided. The method comprises the steps of providing the porous matrix, impregnating the target dsDNA within the porous matrix and amplifying at least one portion of the impregnated target dsDNA within the porous matrix to produce at least one amplicon within the porous matrix. The amplification is performed under isothermal conditions by contacting the impregnated, target dsDNA with a DNA amplification reaction mixture comprising at least one inosine-containing primer, at least one 5′→3′ exonuclease-deficient DNA polymerase having strand displacement activity, at least one nuclease that is capable of nicking a DNA at a residue 3′ to an inosine residue, and dNTP mixture. The DNA amplification may be performed under isothermal conditions without any prior denaturation of the dsDNA to single stranded DNA (ssDNA).

In some embodiments, a method for extracting a genetic material from a biological sample using a nuclease-assisted DNA amplification assay is provided. The biological sample is first contacted with a porous matrix comprising chemicals that lyse the biological sample and preserve the genomic DNA within the porous matrix. At least one portion of the preserved genomic DNA is then amplified within the porous matrix to produce the at least one amplicon within the porous matrix. The at least one amplicon is interrogated or eluted out of the porous matrix for interrogation. The amplification of the preserved genomic DNA is performed within the porous matrix by contacting the preserved genomic DNA within the porous matrix with a DNA amplification reaction mixture comprising at least one inosine-containing primer, at least one 5′→3′ exonuclease-deficient DNA polymerase having strand displacement activity, at least one nuclease that is capable of nicking a DNA at a residue 3′ to an inosine residue, and dNTP mixture. The DNA amplification may be performed under isothermal conditions without any prior denaturation of the dsDNA to single stranded DNA (ssDNA).

### DRAWINGS

These and other features, aspects and advantages of the present invention will become better understood when the following detailed description is read with reference to the accompanying figures.

FIG. 1 illustrates a schematic representation of endonuclease-assisted DNA amplification using inosine-containing primers.

FIG. 2 illustrates a schematic representation of endonuclease-assisted DNA amplification using exonuclease-resistant, inosine-containing primers.

FIG. 3 illustrates enhanced efficiency of endonuclease-assisted DNA amplification using exonuclease-resistant, inosine-containing primers.

FIG. 4 illustrates endonuclease assisted isothermal amplification of a target dsDNA in different porous matrices and in solution.

FIG. 5A illustrates detection limits when endonuclease assisted amplification is performed in solution.

FIG. 5B illustrates detection limits when endonuclease assisted amplification is performed in a PEG-modified nitrocellulose porous matrix.

FIG. 6A illustrates the effects blocking agents and/or other additives on endonuclease assisted isothermal amplification of a target dsDNA in solution.

FIG. 6B illustrates the effects blocking agents and/or other additives on endonuclease assisted isothermal amplification of a target dsDNA in different porous matrices.

### DETAILED DESCRIPTION

To more clearly and concisely describe and point out the subject matter of the claimed invention, the following definitions are provided for specific terms, which are used in the following description and the appended claims.

As used herein, the term “target DNA” refers to a DNA sequence of either natural or synthetic origin that is desired to be amplified in a DNA amplification reaction. The target DNA acts as a template in the nucleic acid amplification reaction. In a DNA amplification reaction, either a portion of a target DNA or the entire region of a target DNA may get amplified by a DNA polymerase to produce one or more amplification products (amplicons). The target DNA may be obtained from a variety of sources, for example, from a biological sample (i.e., a sample obtained from a biological subject) in vivo or in vitro, a food, an agricultural sample, or an environmental sample. The target DNA may be obtained from, but are not limited to, bodily fluid or an exudate (e.g., blood, blood plasma, serum, milk, cerebrospinal fluid, pleural fluid, lymph, tears, sputum, saliva, stool, lung aspirates, throat or genital swabs, or urine), organs, tissues, fractions and sections (e.g., sectional portions of an organ or tissue), cells isolated from a biological subject or from a particular region (e.g., a region containing diseased cells, or circulating tumor cells) of a biological subject, cell fractions, or cultures. The biological sample that contains or suspected to contain the target DNA may be of eukaryotic origin, prokaryotic origin, viral origin, or bacteriophage origin. For example, the target DNA may be obtained from an insect, a protozoa, a bird, a fish, a reptile, a mammal (e.g., rat, mouse, cow, dog, guinea pig, or rabbit), or a primate (e.g., chimpanzee or human). The DNA product generated by another reaction, such as a ligation reaction, a PCR reaction, or a synthetic DNA may also serve as the target DNA. The target DNA may be a circular DNA, a linear DNA or a nicked DNA. The target DNA may be a genomic DNA or a plasmid DNA.

As used herein, the term “DNA amplification reaction mixture” refers to a mixture of reagents that is essential for performing a DNA amplification reaction of a target DNA. The DNA amplification reaction mixture disclosed herein includes, at the minimum, at least one inosine-containing primer, dNTPs, at least one nuclease that is capable of nicking an inosine-containing strand of a dsDNA at a reside 3′ to the inosine residue and at least one DNA polymerase having strand displacement activity. The DNA polymerase may be a 5′→3′ exonuclease-deficient DNA polymerase. It may further include reagents such as buffer(s), salt(s) and other components (e.g., accessory proteins such as single stranded DNA binding protein, denaturant like urea, glycerol, blocking agents like albumin (e.g., BSA) or pyrolidine) that may be required for a DNA amplification reaction.

As used herein, the term “primer” refers to a short linear oligonucleotide that hybridizes to a target DNA to prime a DNA synthesis reaction. The primer may be an RNA oligonucleotide, a DNA oligonucleotide, or a chimeric sequence. The primer may contain natural, synthetic, or modified nucleotides. For example, the primer may comprise naturally occurring nucleotides (G, A, C or T nucleotides) or their analogues. Both the upper and lower limits of the length of the primer sequence may be empirically determined. The lower limit on primer length is the minimum length that is required to form a stable duplex upon hybridization with the target DNA under DNA amplification reaction conditions. Very short primers (usually less than 3 nucleotides long) do not form thermodynamically stable duplexes with target DNA under such hybridization conditions. The upper limit is often determined by the possibility of having a duplex formation in a region other than the pre-determined DNA sequence in the target DNA. Generally, suitable primer lengths are in the range of about 4 nucleotides long to about 40 nucleotides long. In some embodiments the primer ranges in length from 5 nucleotides to 30 nucleotides. The term “forward primer” refers to a primer that anneals to a first strand of the target DNA and the term “reverse primer” refers to a primer that anneals to a complimentary, second strand of the target DNA. Together, a forward primer and a reverse primer are generally oriented on the target DNA sequence in a manner analogous to PCR primers, such that a DNA polymerase can initiate the DNA synthesis resulting in replication of both strands.

As used herein, the term “inosine-containing primer” refers to a primer comprising at least one inosine residue in its sequence. The inosine residue is a 2′-deoxyribonucleoside or 2′-ribonucleoside residue, wherein the nucleobase is a hypoxanthine. Inosine residue may also be an inosine analogue, for example, xanthine structures that result from deamination of guanine. The inosine residue is capable of base pairing with a thymine, an adenine, a cytidine or a uridine residue. Inosine analogues may be a 2′-deoxyribonucleoside or 2′-ribonucleoside wherein the nucleobase includes a modified base such as xanthine, uridine, oxanine (oxanosine), other O-1 purine analogs, N-6-hydroxylaminopurine, nebularine, 7-deaza hypoxanthine, other 7-deazapurines, and 2-methyl purines. The inosine or inosine analogue residue may be positioned near the 3′ terminal end of a primer sequence, often as a penultimate nucleotide at the 3′ end of the primer sequence.

As used herein, the term “exonuclease resistant, inosine-containing primer” refers to an inosine-containing primer sequence that is resistant to the action of an exonuclease enzyme (i.e., not degraded by the exonuclease). In some embodiments, the exonuclease resistant primer may be resistant to both 3′→5′ exonuclease activity and 5′→3′ exonuclease activity. In some embodiments, the exonuclease resistant prime is resistant to 3′→5′ exonuclease activity. The inosine-containing primer may be engineered to make it exonuclease-resistant by chemical modification, for example, by introduction of at least one phosphorothioate linkage at an appropriate position. For example, an inosine-containing primer that contains a phosphorothioate linkage between the 3′ terminal nucleotide and the penultimate residue is resistant to the action of exonuclease in a 3′→5′ direction. Since the 3′→5′ exonuclease digests the nucleic acid in a 3′ to 5′ direction, the phosphorothioate linkage at this position prevents the digestive action of the exonuclease. If the inosine residue is present on the 5′ side of the phosphorothioate, it will be maintained in the primer. Similarly, an inosine-containing primer that contains a phosphorothioate linkage between the 5′ terminal nucleotide and the penultimate residue is resistant to the action of exonuclease in a 5′→3′ direction. When an exonuclease-resistant, inosine containing primer hybridizes with a target DNA and forms double stranded nucleic acid structure, the double stranded structure may be recognized by specific endonucleases resulting in a single stranded nick in the inosine-containing strand. For example, endonuclease V is capable of nicking the inosine-containing strand of a double stranded DNA at a position 3′ to the inosine residue when the exonuclease-resistant inosine-containing primer is hybridized to a target DNA.

As used herein, the term “dNTPs” refers to a mixture of deoxynucleotide triphosphates that act as precursors required by a DNA polymerase for DNA synthesis. Each of the deoxynucleotide triphosphates in a dNTP mixture comprises a deoxyribose sugar, an organic base, and a phosphate in a triphosphate form. A dNTP mixture may include each of the naturally occurring deoxynucleotide triphosphate (e.g., dATP, dTTP, dGTP, dCTP or dUTP). In some embodiments, each of the naturally occurring deoxynucleotide triphosphates may be replaced or supplemented with a synthetic analog, provided however that inosine base may not replace or supplement guanosine base (G) in a dNTP mixture. Each of the deoxynucleotide triphosphates in dNTP may be present in the reaction mixture at a final concentration of 10 μM to 20,000 μM, 100 μM to 1000 μM, or 200 μM to 300 μM.

As used herein, the term “amplicon” refers to nucleic acid amplification products that result from the amplification of a target nucleic acid. Amplicons may comprise a mixture of amplification products (e.g. a mixed amplicon population), several dominant species of amplification products (e.g. multiple, discrete amplicons), or a single dominant species of amplification product. A single species of amplicon may be isolated from a mixed population of amplicons using art-recognized techniques, such as affinity purification or electrophoresis. An amplicon may comprise single-stranded or double-stranded DNA depending on the reaction scheme used. An amplicon may be largely single-stranded or partially double-stranded or completely double-stranded DNA.

The term “mutant endonuclease” or “engineered endonuclease” as used herein refers to an endonuclease enzyme that is generated by genetic engineering or protein engineering, wherein one or more amino acid residues are altered from the wild type endonuclease. The alteration may include a substitution, a deletion or an insertion of one or more amino acid residues. Throughout the specification and claims, the substitution of an amino acid at one particular location in the protein sequence is referred using a notation “(amino acid residue in wild type enzyme) (location of the amino acid in wild type enzyme) (amino acid residue in engineered enzyme)”. For example, a notation Y75A refers to a substitution of a Tyrosine (Y) residue at the 75^{th} position of the wild type enzyme by an Alanine (A) residue (in mutant/engineered enzyme).

The term “conservative variants”, as used herein, applies to both amino acid and nucleic acid sequences. With respect to particular nucleic acid sequences, the term “conservative variants” refers to those nucleic acids that encode identical or similar amino acid sequences (i e, amino acid sequences that have similar physico-chemical properties) and include degenerate sequences. For example, the codons GCA, GCC, GCG, and GCU all encode alanine. Thus, at every amino acid position where an alanine is specified, any of these codons may be used interchangeably in constructing a corresponding nucleotide sequence. Such nucleic acid variants are conservative variants, since they encode the same protein (assuming that is the only alternation in the sequence). One skilled in the art recognizes that each codon in a nucleic acid, except for AUG (sole codon for methionine) and UGG (tryptophan) may be modified conservatively to yield a functionally identical peptide or protein molecule. As to amino acid sequences, one skilled in the art will recognize that alteration of a polypeptide or protein sequence via substitutions, deletions, or additions of a single amino acid or a small number (typically less than about ten) of amino acids may be a “conservative variant” if the physico-chemical properties of the altered polypeptide or protein sequence is similar to the original. In some cases, the alteration may be a substitution of one amino acid with a chemically similar amino acid. Examples of conservative variants include, but not limited to, the substitution of one hydrophobic residue (e.g., isoleucine, valine, leucine or methionine) for one another; or the substitution of one polar residue for another (e.g., the substitution of arginine for lysine, glutamic for aspartic acid, or glutamine for asparagine) and the like. Genetically encoded amino acids generally may be divided into four families: (1) acidic: aspartate, glutamate; (2) basic: lysine, arginine, histidine; (3) nonpolar: alanine, valine, leucine, isoleucine, proline, phenylalanine, methionine, tryptophan; and (4) uncharged polar: glycine, asparagine, glutamine, cysteine, serine, threonine, tyrosine.

The examples of the methods relate to endonuclease-assisted DNA amplification (referred to as “Ping Pong” amplification) of a target dsDNA on a porous matrix. The endonuclease-assisted DNA amplification is generally an inosine-mediated amplification, wherein the endonuclease is capable of nicking an inosine-containing strand of a dsDNA. The target dsDNA is amplified as such without any prior denaturation to ssDNA molecules. The target dsDNA, which is impregnated on the porous matrix, is amplified within the porous matrix to produce at least one amplicon based on the target dsDNA. The target dsDNA is not eluted out of the porous matrix prior to or after the amplification reaction. The amplification reaction happens inside the porous matrix and not in solution and the amplicons are generated within the porous matrix. The amplicons may be a single stranded or a double-stranded DNA, often extending to the end of the template strand. Ping Pong amplification may use about 2 to 20 or more total numbers of nested primers to amplify a target dsDNA template. The generated amplicons may be subsequently detected within the porous matrix using a variety of techniques or they may be eluted out of the porous matrix using appropriate eluents.

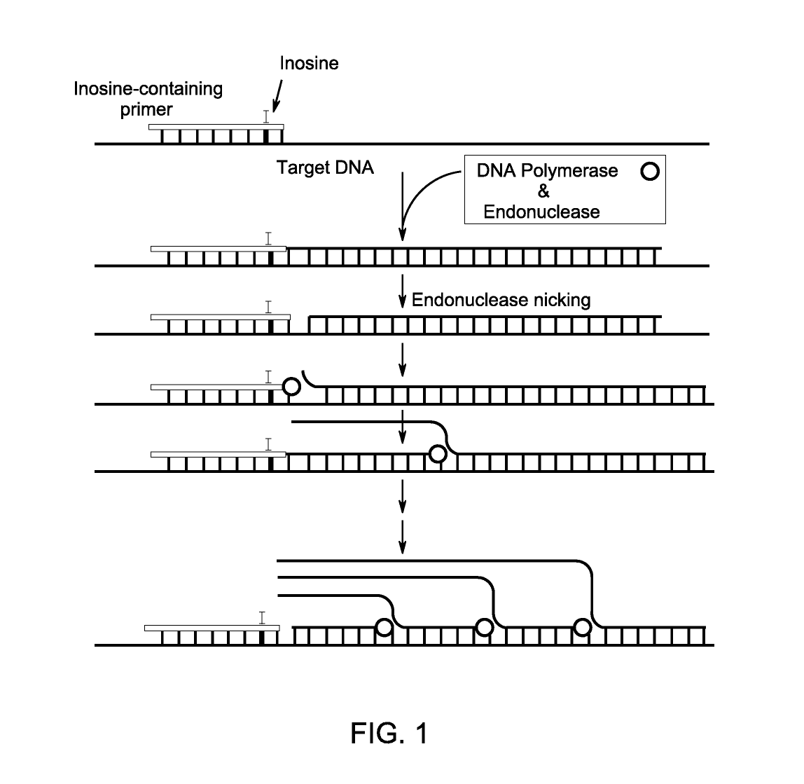

FIG. 1 depicts a schematic representation of an embodiment of endonuclease-assisted target DNA amplification. The target DNA is amplified using an inosine-containing primer, a DNA polymerase (e.g., a strand displacement polymerase) and dNTPs in presence of an endonuclease (e.g., endonuclease V) that is capable of nicking an inosine-containing strand of a dsDNA at a location 3′ to the inosine residue. Inosine residue in the primer may base pair with a cytidine residue or a thymidine residue in the target DNA, wherein hypoxanthene substitutes for a guanine to complement a cytosine, or substitutes for an adenine to complement a thymine. Upon binding of the inosine-containing primer to the target DNA, the DNA polymerase (e.g., a 5′→3 exonuclease-deficient Bst DNA polymerase) extends the inosine-containing primer thereby generating a dsDNA (primer extension product). Generation of the dsDNA in turn generates a nicking site for the endonuclease, which is capable of creating a single stranded nick at the inosine-containing strand of a dsDNA at a position 3′ to the inosine residue. The endonuclease nicks the inosine-containing strand of this double-stranded DNA. Nicking creates a new DNA synthesis initiation site for the DNA polymerase. The DNA polymerase binds to this initiation site and further elongates the nicked primer. DNA polymerases use dNTP mixture to add nucleotides to the 3′ hydroxyl end of the nick creating a new DNA strand (amplicon) complementary to the target DNA's template strand. This elongation step once again creates a nicking site for the endonuclease. Since the DNA polymerase has strand displacement activity, it displaces a ssDNA product while it re-creates the double-stranded primer extension product. Thus the elongation by the DNA polymerase followed by nicking by the endonuclease such as endonuclease V gets repeated multiple times until any one of the essential DNA amplification reagents in the DNA amplification reaction mixture is exhausted. In each cycle, the strand displacement DNA polymerase employed in these reactions displaces the complementary strand that was generated in the previous cycle. The steps of hybridization, elongation, nicking and further elongation may occur substantially simultaneously. This cycle repeats, synthesizing multiple single strands of DNA complementary to the downstream portion of the target DNA template.

The inosine-containing primer may be a forward primer or a reverse primer or a mixture of both. Amplicons may be generated using a single inosine-containing primer, paired inosine-containing primers, or nested-paired inosine-containing primers. The inosine-containing primer may demonstrate a melting temperature of 25° C. to 80° C., 30° C. to 65° C., or 40° C. to 55° C. in the reaction mixture. In some embodiments, the inosine-containing primer demonstrates a melting temperature of 50° C. in the reaction mixture having a salt concentration of about 6 mM. The inosine-containing primer(s) are engineered to have a nucleotide sequence that is complementary, in the Watson-Crick sense, to a pre-determined sequence, which is present in the target DNA template.

In general, the inosine analogue residue of the inosine-containing primer is positioned away from the 5′ end of the prime such that the primer remains annealed to the target DNA after nicking by the endonuclease (e.g., the length of the nicked primer is sufficient to enable binding to the target DNA under the nucleic acid amplification reaction conditions). In some embodiments, the inosine nucleotide in the inosine-containing primer may be positioned at least 4 nucleotides, at least 5 nucleotides, or at least 10 nucleotides downstream of the 5′ end of the inosine-containing primer. In some embodiment, the inosine-containing primer may comprise more than one inosine residue or inosine analogues. For example, inosine may be present at both the penultimate 3′ residue and ultimate 3′ residue (e.g., NNNNNIIN). In some embodiments, an inosine analogue may be substituted for the inosine of the inosine-containing primer.

An endonuclease such as endonuclease V may be used as for the DNA amplification reaction. Endonuclease V is a repair enzyme that recognizes DNA containing inosines (or inosine analogues) and hydrolyzes the second or third phosphodiester bonds 3′ to the inosine (i.e., specifically nicks a DNA at a position two nucleotides 3′ to an inosine nucleotide, about 95% the second phosphodiester bond and about 5% the third phosphodiester bond) leaving a nick with 3′-hydroxyl and 5′-phosphate. When the target DNA is double stranded, the Endonuclease V nick occurs in the strand comprising the inosine residue.

Endonuclease-assisted DNA amplification as illustrated in FIG. 1 may be varied, for example, by employing additional primers, additional enzymes, additional nucleotides, stains, dyes, or other labeled components. For example, each of the naturally occurring deoxynucleotides in a dNTP mixture may be replaced or supplemented with a synthetic analogue, provided however that deoxyinosine triphosphate (dITP) may not replace or supplement dGTP in the dNTP mixture. With a single, forward primer, the rate of synthesis of complimentary copies of target DNA is relatively constant, resulting in a steady, linear increase in the number of complimentary copies with time. Multiple primers may be included in the reaction mixture to accelerate the amplification process. Paired primers comprising a forward primer and a reverse primer may be included in the reaction mixture for generating both the plus and minus strands. For example, when a reverse primer (a primer that anneals to the generated complementary strand ((+) strand) to further generate a (−) strand in the reverse direction) that anneals to the complementary strand of target DNA at a defined distance from the forward primer is added, the amplification process is accelerated. Since the targets for each of these primers would be present in the original template, both strands would be amplified in the two primer scheme (“Ping product” being the amplicon of the forward primer and the “Pong product” being the amplicon of the reverse primer). The inclusion of multiple paired primers may improve the relative percentage of a discrete product in the reaction mixture.

In some embodiments, a method of producing at least one amplicon based on a target double stranded DNA within a porous matrix using an endonuclease-assisted DNA amplification is provided. The method comprises the steps of (a) providing the porous matrix, (b) impregnating the target double stranded DNA within the porous matrix, (c) contacting the impregnated, target double stranded DNA with a DNA amplification reaction mixture comprising at least one inosine-containing primer, at least one 5′→3′ exonuclease-deficient DNA polymerase having strand displacement activity, at least one nuclease that is capable of nicking a DNA at a residue 3′ to an inosine residue, and dNTP mixture, and (d) amplifying at least one portion of the impregnated target double stranded DNA within the porous matrix using the DNA amplification reaction mixture of step (c) to produce the at least one amplicon within the porous matrix. The nuclease may be an endonuclease that is capable of nicking the inosine-containing strand of a double stranded DNA at a residue 3′ to an inosine residue. In some embodiments, an endonuclease V may be used in the DNA amplification reaction. The endonuclease V may be a wild type endonuclease or a mutant endonuclease.

The DNA amplification reaction on the porous matrix is performed under isothermal conditions. The reaction temperature during an isothermal amplification reaction condition may range 1° C., 5° C., or 10° C. from a set temperature. In some embodiments, the target dsDNA is amplified within the porous matrix at 45° C. (±1° C.). Thermally stable endonucleases and DNA polymerases may be used depending upon the reaction temperature of DNA amplification reaction.

The impregnated dsDNA in the porous matrix is amplified within the porous matrix without any prior denaturation step (e.g., thermal denaturation). Further, the dsDNA is not eluted out of the porous matrix prior to amplification, and thus amplification happens in the porous matrix and not in the solution in a tube or well. The primers, either alone or in combination may increase the chances to strand invade to create a “D-loop” that initiates the endonuclease-assisted DNA amplification. Once the target DNA amplification is over, the amplicons may be detected within the porous matrix to determine the presence, absence or quantity of a particular amplicon and/or to detect the reaction kinetics of DNA amplification. Further, the amplicon may subsequently be eluted out of the porous matrix post amplification. The impregnation of the target dsDNA within the porous matrix may be performed by any of the known methods. The impregnation may be done via chemisorption or physiosorption. For example, a porous matrix (e.g., FTA™ paper, GE Healthcare) that contains impregnated lysing agents (e.g., detergents, urea) may be used to lyse a biological sample in the porous matrix. The detergent may be an ionic detergent such as sodium dodecyl sulfate (SDS). The porous matrix may further contain chemicals that stabilize a genomic DNA (e.g., an anti-oxidant, chaotrope). Thus the genomic DNA in a biological sample may be impregnated in the porous matrix by contacting a biological sample with a porous matrix, which lyses the biological sample and preserve the genomic DNA. When the porous matrix contains a detergent, an amplification mixture comprising a detergent sequestering agent may be used for DNA amplification. The detergent sequestering agent sequesters the ionic surfactant that is present in the porous matrix, which may otherwise inhibit the DNA amplification reaction. Suitable detergent sequestering agent includes, but are not limited to, alpha-cyclodextrins and polyamines. In some embodiments, the genomic DNA may not leach out of the porous matrix due to its high molecular weight. However, after amplification of at least one portion of the impregnated genomic DNA, the amplicon, due to its smaller sizes, may be eluted to a different area of the porous matrix or may be eluted out of the porous matrix by using appropriate eluents (e.g., TE buffer).

The porous matrix may be selected so that they are capable of holding the target dsDNA within its porous structures. The efficient impregnation of target dsDNA may depend on a variety of factors including the pore size, biocompatibility and the molecular weight of the dsDNA. Suitable porous matrixes that may be used include, but are not limited to, a cellulose membrane, a nitrocellulose membrane, a cellulose acetate membrane, a nitrocellulose mixed ester membrane, a glass fiber, a polyethersulfone membrane, a nylon membrane, a polyolefin membrane, a polyester membrane, a polycarbonate membrane, a polypropylene membrane, a polyvinylidene difluoride membrane, a polyethylene membrane, a polystyrene membrane, a polyurethane membrane, a polyphenylene oxide membrane or a poly(tetrafluoroethylene-co-hexafluoropropylene) membrane. The porous matrix may be modified (e.g., coated) with suitable materials to alter the characteristics of the porous matrix. For example, the porous matrix may be coated with an alkyl(oligooxyalkylene) group (e.g., an R—(O—CH₂—CH₂—)ₙ group) to reduce the active binding (e.g., specific binding) of proteins to the porous matrix. In some embodiments, the porous matrix may be a cellulose membrane or a nitrocellulose membrane. In some other embodiments, the porous matrix is a polyethyleneglycol-modified cellulose membrane or polyethyleneglycol-modified nitrocellulose membrane with reduced protein binding capabilities.

In some embodiments, the porous matrix may be FF60 nitrocellulose (GE Healthcare), PEGMA 300 grafted nitrocellulose (NC-PEG), 903 cellulose (GE Healthcare), PEGMA 300 grafted 903 cellulose (903-PEG), 31-ETF cellulose (GE Healthcare), Fusion 5 (GE Healthcare), Glass Fiber (Standard 17, GF/F, both from GE Healthcare), or Quartz (QMA, from GE Healthcare). Coating and grafting of porous cellulose substrates (e.g., 903, 31ETF) or nitrocellulose (FF60, FF80HP) may be performed in an aqueous solution containing 10% PEGMA 300 (polyethylene glycol methyl ether methacrylate, Number average molecular weight (Mₙ): 300, from Sigma Aldrich) and 30% Tween 20, followed by electron beam irradiation of the coated substrate. Electron beam (e-beam) irradiation creates free radicals on the membrane surface which initiates polymerization of the methacrylate monomers, and as a result, the PEG moieties are permanently introduced to the membranes. After the e-beam treatment, the treated membranes may be washed with water to remove co-solvent Tween 20 and ungrafted PEG species and then dried. For example, the polyethyleneglycol-modified cellulose membrane is fabricated through e-beam irradiation of the cellulose membrane in presence of an active compound containing a polyethyleneglycol group (e.g., polyethyleneglycol methyl ether methacrylate). Polyethyleneglycol-modified nitrocellulose membrane is fabricated through e-beam irradiation of the nitrocellulose membrane in presence of an active compound containing a polyethyleneglycol group (e.g., polyethyleneglycol methyl ether methacrylate).

The DNA amplification reaction mixture may further comprise one or more of reagents that enhance or assist DNA amplification reaction such as buffers, single strand DNA binding protein (e.g., E. coli SSB, T4 gene 32 protein (T4 g32p), T7 gene 2.5 protein, Ncp7, recA, or combinations thereof), topoisomerase, formamide, ethylene glycol, reducing agents, alpha-cyclodextrin, or Ficoll. Any buffers (e.g., Tris buffer, HEPES buffer) that result in a reaction pH between 6 and 9 may be used for the DNA amplification reaction. In some embodiments, the pH of the DNA amplification reaction mixture is about 8.0. In some embodiments, buffers that enhance DNA stability (e.g., HEPES) may be used. Thermo-labile buffers such as Tris-Borate, HEPES, and MOPS buffers may be disfavored in some specific DNA amplification reactions. In some specific embodiments, the DNA amplification reaction buffer may comprise 25 mM Tris-Borate; 5 mM MgCl₂; 0.01% Tween; and 20% ethylene glycol. The DNA amplification reaction mixture may further include one or more of blocking agents (e.g., bovine serum albumin (BSA), human serum albumin, bactopeptone, casein) and/or surfactants (e.g., non-ionic detergents, for example 0.01% of Tween 20). The non-specific binding of proteins in the DNA amplification reaction mixture (e.g., DNA polymerase) on to the porous matrix may render it unavailable for DNA amplification reaction. Inclusion of blocking agents and/or detergents in the DNA amplification reaction mixtures reduces or eliminates the non-specific binding of proteins or other reagents to the porous matrix. In some embodiments, the surfactant may be a non-ionic detergent selected from Tween-20, NP-40, Triton-X-100, or combinations thereof. In some embodiments, 0.05% NP-40 and 0.005% Triton X-100 is used for the reaction. The DNA amplification reaction mixture may further include at least one topoisomerase (e.g., a type 1 topoisomerase). The topoisomerase may be present in the reaction mixture at a final concentration of at least 0.1 ng/μL. The single stranded DNA binding protein may be present in the reaction mixture at a final concentration of at least 0.1 ng/μL. The DNA amplification reaction mixture may also include one or more reducing agents such as dithiothreitol (DTT), 2-mercaptoethanol (βME), Tris(carboxyethyl) phosphine (TCEP), or 2-mercaptoethylamine (MEA) that reduces the oxidation of enzymes in the reaction mix and improves the quality and yield of the amplicons produced. In some embodiments, the DNA amplification mixture further includes one or more of blocking agents such as albumin (e.g., BSA), powdered milk, gelatin, casein, or bactopeptone. The DNA amplification reaction may be performed in an unmodified porous substrate in presence of a blocking agent. The DNA amplification reaction may be performed in an modified porous substrate either in presence or in absence of a blocking agent.

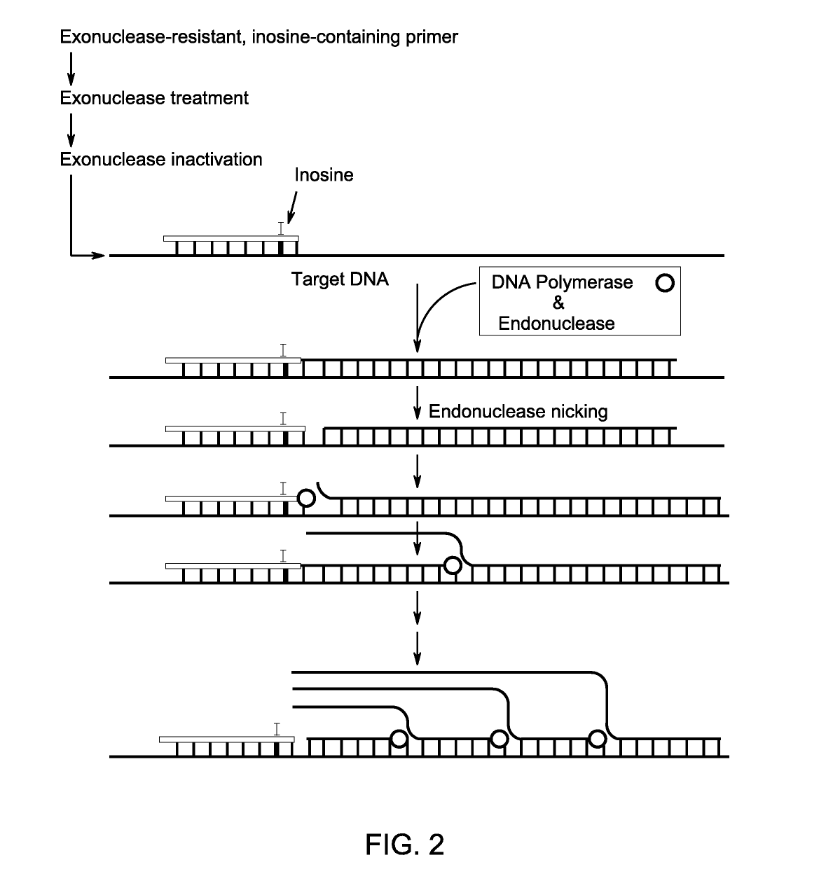

The primer employed for the DNA amplification reaction in the porous matrix may be exonuclease-resistant. FIG. 2 depicts a schematic representation of an embodiment of the endonuclease-assisted DNA amplification reaction using exonuclease-resistant, inosine-containing primer. Prior to the generation of the DNA amplification mixture, the primer solution containing the exonuclease-resistant, inosine-containing primer, may be decontaminated by treating with an appropriate exonuclease to remove any contaminating nucleic acid. The decontamination of the primer solution is often achieved by incubating the primer solution with an exonuclease and a divalent cation to allow the exonuclease to render the contaminating nucleic acid inert. A single exonuclease or a combination of exonucleases may be used to decontaminate the primer solution. Suitable exonucleases include, but are not limited, exonuclease I, exonuclease III, exonuclease VII, T7 gene-6 exonuclease, spleen exonuclease, T5 D15 exonuclease or lambda exonuclease. In one embodiment, a combination of exonuclease I and exonuclease III is used for decontaminating the primer solution. After the decontamination reaction, the added exonuclease in the primer solution may be inactivated. If inactivation is not performed after the decontamination reaction, the quantity of exonuclease may be selected such that it does not interfere with the subsequent DNA amplification reaction in the porous matrix. In some embodiments, extender templates, which are specific primer sequences (e.g., primers that contain additional 5′ sequences that allow for additional sequence (e.g., a promoter sequence or a restriction endonuclease site specific sequence or a novel primer binding site sequence) to be added to the end of hybridized amplicon DNA) may be annealed at the 3′ end of the amplicon.

The exonuclease-resistant inosine-containing primer may comprise at least one nucleotide that makes the primer resistant to degradation by an exonuclease, particularly by a 3′→5′ exonuclease. For example, an exonuclease resistant primer may possess one or more phosphorothioate linkages between nucleotides in the sequence (e.g., NNNNN*N*N*I*N or N*NNNN*N*N*I*N). The modified nucleotide is commonly a 3′-terminal nucleotide of the primer sequence having a penultimate inosine residue (e.g., (NNN)ₙNI*N or (NNN)ₙNI*I where * represents a phosphorothioate bond between the nucleotides and the integer value of n may range depending on the length of the primer used, for example, the value of n may range from 0 to 13). However, in some embodiments, the primer could have the modified nucleotide as the inosine residue (e.g., NNNNNN*IN). In some embodiments, the modified nucleotide may be located at a position other than the 3′-terminal position provided that the primer sequence contains at least one inosine residue located next to the modified residue (e.g., NNNNI*NNNN or NNNN*INNNN). When the modified nucleotide is located at positions other than the 3′-terminal end of a primer sequence, the 3′-terminal nucleotide of said primer may be removed by the 3′→5′ exonuclease activity. Other nucleotide modifications known in the art that make a nucleotide sequence resistant to an exonuclease may be used as well.

Any of the DNA polymerases known in the art may be employed for DNA amplification. DNA polymerases suitable for use in the inventive methods may demonstrate one or more of the following characteristics: strand displacement activity, the ability to initiate strand displacement from a nick, and/or low degradation activity for single stranded DNA. In some embodiments, the DNA polymerase employed may be devoid of one or more exonuclease activities. For example, the DNA polymerase may be a 3′→5′ exonuclease-deficient DNA polymerase or the DNA polymerase may lack 5′→3′ exonuclease activity. In some embodiments, the DNA polymerase may lack both 3′→5′ and 5′→3′ exonuclease activities (i.e., an exo (−) DNA polymerase). Exemplary DNA polymerases useful for the methods include, without limitation, 5′→3′ exonuclease-deficient Klenow, 5′→3′exonuclease-deficient Bst DNA polymerase (the large fragment of Bst DNA polymerase), 5′→3′ exonuclease-deficient delta Tts DNA polymerase, 5′→3′ exonuclease-deficient T7 polymerase exo (−) Klenow, or exo(−) T7 DNA polymerase (Sequenase™). In some embodiments, a combination of DNA polymerases may be used.

DNA polymerase enzymes typically require divalent cations (e.g., Mg⁺², Mn⁺², or combinations thereof) for DNA synthesis. Accordingly, one or more divalent cations may be added to the DNA amplification reaction mixture. For example, MgCl₂ may be included in the reaction mixture at a concentration range of 2 mM to 6 mM. Higher concentrations of MgCl₂ may be preferred when high concentrations (e.g., greater than 10 pmoles, greater than 20 pmoles, or greater than 30 pmoles) of inosine-containing primer are included in the reaction mixture.

In some embodiments, a mutant endonuclease V is included in the DNA amplification reaction mixture to nick the inosine-containing double stranded DNA. The mutant E. coli endonuclease may be a Y75A mutant E. coli endonuclease V corresponding to SEQ ID NO: 2. This mutant is generated by replacing the Tyrosine (Y) residue at the 75^{th} position of a wild type E. coli endonuclease V (SEQ ID NO: 1) with an Alanine (A) residue. In some embodiments, a mutant Afu endonuclease Y74A (SEQ ID NO: 4) and/or its conservative variants is employed. The mutant Y74A Afu endonuclease is generated by substituting a Tyrosine (Y) residue at the 75^{th} position of a wild type Afu endonuclease V (SEQ ID NO: 3) with an alanine (A) residue. In some embodiments, a Y80A mutant of Termotoga maritima (Tma) endonuclease V (SEQ ID NO: 6) and/or its conservative variants is included the DNA amplification reaction mixture.

In some embodiments, a rationally designed, mutant endonuclease V enzyme is employed that has increased substrate binding, increased nicking efficiency, increased nicking specificity and/or increased nicking sensitivity. A mutant endonuclease V may also be designed such that the substrate binding is reversible. The mutant endonuclease V enzyme may then support repeated nicking by each enzyme, whereas the corresponding wild type enzyme may be capable of only a single round (or a few limited rounds) of nicking (for example, the wild type E. coli endonuclease V (SEQ ID NO: 1) remains bound to the DNA after nicking). Such mutant endonuclease V may be used in a reaction mixture in less than stoichiometric quantities to effect a nicking reaction. In some embodiments, a conservative variant of the mutant endonuclease V may be used for the DNA amplification reaction. For example, further alteration of a mutant endonuclease V via substitution, deletion, and/or addition of a single amino acid or a small number (typically less than about ten) of amino acids may be a “conservative variant” if the physico-chemical properties of the altered mutant endonuclease V is similar to the original mutant endonuclease V. In some cases, the alteration may be a substitution of one amino acid with a chemically similar amino acid.

The mutant endonuclease V may have a higher efficiency than the wild type endonuclease V to nick the inosine-containing strand of the double stranded DNA when the inosine is paired with cytosine or thymine. Further, a mutant endonuclease V may preferentially nick an inosine-containing strand of a double stranded DNA than an inosine-containing single stranded DNA. For example, Y75A E. coli mutant endonuclease V (SEQ ID NO: 2) nicks a double stranded DNA comprising an inosine residue better than a single stranded DNA comprising an inosine residue. In contrast, Y80A Tma mutant endonuclease V (SEQ ID NO: 6) nicks a single stranded DNA comprising an inosine residue better than a double stranded DNA comprising an inosine residue. Some mutant endonucleases may nick structures other than DNA sequences containing inosine residue while some others may be very specific to inosine-containing DNA sequences. For example, Tma and Afu endonucleases (SEQ ID NO: 3 and SEQ ID NO: 5) do not nick structures such as flaps and pseudo Y structures. In some embodiments, when there are multiple inosine residues in a double stranded DNA, the employed endonuclease V mutant may preferentially nick (often 1 or 2 nucleotides 3′ to the inosine residue) the inosine residue that is paired with a cytosine residue than the inosine residue that is paired with a thymine residue. In some aspects, the endonuclease V mutant may nick a double stranded DNA containing base pair mismatches. The nicking may happen at the location of the base pair mismatch or at a location 3′ to the base pair mismatch that is separated by one or more bases.

In some embodiments, a heat stable endonuclease V is used for the DNA amplification reaction. For example, Y75A E. coli endonuclease V mutant is inactivated by incubation at temperatures above 50° C., whereas it retains its enzymatic activity at 37-49° C. Archaeoglobus fulgidus (Afu) endonuclease V (both wild type (SEQ ID NO: 3) and Y75A mutant (SEQ ID NO: 4)) or Tma endonuclease V (both wild type (SEQ ID NO: 5) and Y80A mutant (SEQ ID NO: 6)) are generally more thermo stable than the E. coli endonuclease V (both wild type (SEQ ID NO: 1) and Y75A mutant (SEQ ID NO: 2)). In some embodiments where strand displacement DNA synthesis by DNA polymerase may be increased by incubation at an elevated temperature, an endonuclease V which functions at high temperature (e.g., 45-80° C.) may be used.

Table 1 provides the sequences of wild type endonucleases and mutant endonuclease V enzymes.

TABLE 1
Sequences of wild type endonucleases, mutant endonucleases,
template DNAs, and various primers

| | Ref. No. | Sequence (N-term - C-term; 5′→3′) | Length |
|---|---|---|---|
| Wide Type E. | SEQ ID | MIMDLASLRAQQIELASSVIREDRLDKD | 225 |
| coli | NO: 1 | PPDLIAGADVGFEQGGEVTRAAMVLLK | |
| endonuclease V | | YPSLELVEYKVARIATTMPYIPGFLSFRE | |
| | | YPALLAAWEMLSQKPDLVFVDGHGISH | |
| | | PRRLGVASHFGLLVDVPTIGVAKKRLCG | |
| | | KFEPLSSEPGALAPLMDKGEQLAWVWR | |
| | | SKARCNPLFIATGHRVSVDSALAWVQR | |
| | | CMKGYRLPEPTRWADAVASERPAFVRY | |
| | | TANQP | |
| Y75A mutant E. | SEQ ID | MIMDLASLRAQQIELASSVIREDRLDKD | 225 |
| coli | NO: 2 | PPDLIAGADVGFEQGGEVTRAAMVLLK | |
| endonuclease V | | YPSLELVEYKVARIATTMPAIPGFLSFRE | |
| | | YPALLAAWEMLSQKPDLVFVDGHGISH | |
| | | PRRLGVASHFGLLVDVPTIGVAKKRLCG | |
| | | KFEPLSSEPGALAPLMDKGEQLAWVWR | |
| | | SKARCNPLFIATGHRVSVDSALAWVQR | |
| | | CMKGYRLPEPTRWADAVASERPAFVRY | |
| | | TANQP | |
| Wild Type Afu | SEQ ID | MLQMNLEELRRIQEEMSRSVVLEDLIPL | 221 |
| endonuclease V | NO: 3 | EELEYVVGVDQAFISDEVVSCAVKLTFP | |
| | | ELEVVDKAVRVEKVTFPYIPTFLMFREG | |
| | | EPAVNAVKGLVDDRAAIMVDGSGIAHP | |
| | | RRCGLATYIALKLRKPTVGITKKRLFGE | |
| | | MVEVEDGLWRLLDGSETIGYALKSCRR | |
| | | CKPIFISPGSYISPDSALELTRKCLKGYKL | |
| | | PEPIRIADKLTKEVKRELTPTSKLK | |
| Y74A mutant | SEQ ID | MLQMNLEELRRIQEEMSRSVVLEDLIPL | 221 |
| Afu | NO: 4 | EELEYVVGVDQAFISDEVVSCAVKLTFP | |
| endonuclease V | | ELEVVDKAVRVEKVTFPAIPTELMFREG | |
| | | EPAVNAVKGLVDDRAAIMVDGSGIAHP | |
| | | RRCGLATYIALKLRKPTVGITKKRLFGE | |
| | | MVEVEDGLWRLLDGSETIGYALKSCRR | |
| | | CKPIFISPGSYISPDSALELTRKCLKGYKL | |
| | | PEPIRIADKLTKEVKRELTPTSKLK | |
| Wild Type Tma | SEQ ID | MDYRQLHRWDLPPEEAIKVQNELRKKI | 225 |
| endonuclease V | NO: 5 | KLTPYEGEPEYVAGVDLSFPGKEEGLAV | |
| | | IVVLEYPSFKILEVVSERGEITFPYIPGLL | |
| | | AFREGPLFLKAWEKLRTKPDVVVFDGQ | |
| | | GLAHPRKLGIASHMGLFIEIPTIGVAKSR | |
| | | LYGTFKMPEDKRCSWSYLYDGEEIIGCV | |
| | | IRTKEGSAPIFVSPGHLMDVESSKRLIKA | |
| | | FTLPGRRIPEPTRLAHIYTQRLKKGLF | |
| Y80A mutant | SEQ ID | MDYRQLHRWDLPPEEAIKVQNELRKKI | 225 |
| Tma | NO: 6 | KLTPYEGEPEYVAGVDLSFPGKEEGLAV | |
| endonuclease V | | IVVLEYPSFKILEVVSERGEITFPAIPGLL | |
| | | AFREGPLFLKAWEKLRTKPDVVVFDGQ | |
| | | GLAHPRKLGIASHMGLFIEIPTIGVAKSR | |
| | | LYGTFKMPEDKRCSWSYLYDGEEIIGCV | |
| | | IRTKEGSAPIFVSPGHLMDVESSKRLIKA | |
| | | FTLPGRRIPEPTRLAHIYTQRLKKGLF | |

The amplicons produced by various embodiments of the present DNA amplification methods may be determined qualitatively or quantitatively by any of the existing techniques. The amplicons may be detected either within the porous matrix or outside of the porous matrix. For example, for a qualitative or quantitative assay, terminal-phosphate-labeled ribonucleotides may be used in combination with a phosphatase during/after DNA amplification reaction for color generation. In such embodiments, the terminal phosphate may be protected from dephosphorylation by using terminal-phosphate methyl esters of dNTPs or deoxynucleoside tetraphosphates.

In some embodiments, a method for extracting a genetic material from a biological sample via amplification of the genetic material is provided. The genetic material may be an amplicon derived from a portion of the genomic DNA, for example, via whole genome amplification. In some embodiments, the method comprises the steps of lysing the biological sample in a porous matrix by contacting the biological sample with a porous matrix comprising chemicals that can lyse the biological sample and preserve the genomic DNA within the porous matrix. The porous matrix may be a solid matrix and lysing chemicals may be impregnated in the porous matrix in dried formats (e.g., a paper strip impregnated with lysing chemicals). The lysing chemicals may include, for example, salts, detergents, chaotropes, reducing agents, anti-oxidants, chelating agents or buffers. For example, the porous substrate may be impregnated with one or more of sodium dodecyl sulfate (SDS), urea, guanidinium chloride, or arginine. Upon contacting the biological sample with the porous matrix, the impregnated chemicals lyse the biological sample (e.g., cell lysis) within the porous matrix and preserve/stabilize the genomic DNA within the porous matrix. The preserved genomic DNA may be stored for longer periods in the porous matrix, if needed.

The preserved genomic DNA may be amplified within the porous matrix under isothermal conditions by using the above-disclosed endonuclease-assisted DNA amplification methods. For example, in one embodiment, this may be achieved by contacting the preserved genomic DNA with a DNA amplification reaction mixture comprising at least one inosine-containing primer, at least one 5′→3′ exonuclease-deficient DNA polymerase having strand displacement activity, at least one nuclease that is capable of nicking a DNA at a residue 3′ to an inosine residue, and dNTP mixture. The nuclease may be an endonuclease such as endonuclease V or a mutant endonuclease V. At least one portion of the preserved genomic DNA may be amplified under isothermal conditions within the porous matrix using the DNA amplification reaction mixture to produce the at least one amplicon within the porous matrix.

The porous matrix may be selected such that high molecular weight DNA (e.g., genomic DNA) does not elute out of the porous matrix. For example, after lysing the biological sample within the porous matrix, the porous matrix may be washed with appropriate eluents (e.g., a TE buffer) to remove one or more chemicals that are used for lysing the biological sample and/or preserving the genetic material. The washing of the porous matrix may be useful especially if the lysis chemicals include any components that may interfere with the endonuclease-assisted DNA amplification reaction. After the amplification, the produced amplicon has comparatively low molecular weight DNA. These amplicons may then be eluted out of the porous matrix by using appropriate eluents.

Unlike other isothermal DNA amplification reactions, wherein a bumper primer that extends and knock of the first primer to make that first extension product single stranded, endonuclease-assisted DNA amplification repeatedly copies the target dsDNA. The target dsDNA does not move or elute out from the porous matrix due to its size. However, the generated, smaller amplicons may be eluted out of the porous matrix.

Practice of the invention will be still more fully understood from the following examples, which are presented herein for illustration only and should not be construed as limiting the scope of the present invention as defined by the appended claims.

### EXAMPLES

Some abbreviations used in the examples section are expanded as follows: “mg”: milligrams; “ng”: nanograms; “pg”: picograms; “fg”: femtograms; “mL”: milliliters; “mg/mL”: milligrams per milliliter; “mM”: millimolar; “mmol”: millimoles; “pM”: picomolar; “pmol”: picomoles; “μL”: microliters; “min.” minutes and “h.”: hours.

All melting temperature values provided herein are determined according to the formula, 100.5+(41*(yG+zC−16.4)/(wA+xT+yG+zC))−(820/(wA+xT+yG+zC))+16.6*LOG 10([Na+]+[K+])−0.56(% EG)−0.32(% G)−0.62(% F) where w, x, y and z refer to the number of adenosine, cytosine, guanosine and thymidine residues, respectively, contained in the primer, Na+ refers to the sodium concentration (mM), K+ refers to the potassium concentration (mM), EG refers to the ethylene glycol concentration (%), G refers to the glycerol concentration (%), and F refers to the formamide concentration (%).

TRIS-HCl and Tween 20 were obtained from Sigma Aldrich; dNTPs were obtained from GE Healthcare; and NaCl was obtained from Ambion. Volumes shown in microliters unless otherwise indicated. Amplicons may be visualized and/or quantified using any of art-recognized techniques (e.g., electrophoresis to separate species in a sample and observe using an intercalating dye such as ethidium bromide, acridine orange, or proflavine). Amplicon production may also be tracked using optical methods (e.g., ABI Series 7500 Real-Time PCR machine) and an intercalating dye (e.g., SYBR Green I). The amplicons produced in the following examples were visualized using electrophoresis or optical techniques.

HET Buffer is 10 mM HEPES Buffer, pH 8, 0.1 mM EDTA and 0.1% (v/v) Tween 20. 10× denaturation buffer is 100 mM HEPES Buffer, pH 8.0, 1 mM EDTA, 0.1% (v/v) Tween 20 and 10 mg/ml BSA. 10× Reaction Buffer is 150 mM HEPES Buffer, pH 8, 30 mM magnesium chloride, 1 mM manganese sulphate, 2.5 mM dATP, 2.5 mM dCTP, 2.5 mM dGTP, 2.5 mM dTTP, 50 mM ammonium sulfate, 10 mM TCEP and 0.1% (v/v) Tween 20. Enzyme Dilution Buffer is 10 mM HEPES, pH 8, 1 mM TCEP, 0.5 mM EDTA, 0.01% (v/v) Tween 20 and 50% (v/v) glycerol. 5% (w/v) Ficoll 400 is equivalent to 5 g/100 ml of water.

### Example 1

### Endonuclease-Assisted Isothermal Amplification of a Template DNA

A completed Ping Pong amplification reaction is composed of three separate parts termed either 1) Denaturation 2) Enzyme Mix or 3) Reaction Mix. The different parts are prepared in the order indicated and eventually combined to start the reaction. Denaturation involves mixing the DNA template and primers in a buffered chemical denaturant to separate the template strands and allow primer annealing. No heating is required to denature the template. Once the Denaturation has been formulated, an Enzyme Mix containing all the necessary proteins is prepared in a buffered glycerol solution. For example, the Enzyme mix may include a mutant Endo V, a Bst DNA Polymerase and a single stranded DNA binding protein (SSB). The Enzyme Mix is then combined with dNTPs and requisite divalent cations to prepare the Reaction Mix. Finally, the Denaturation and Reaction Mix are mixed and immediately placed at 45° C. to start amplification.

The Denaturation is prepared by mixing 1.0 μL of 10× Denaturation Buffer, 1.0 μL of template at the appropriate concentration, 1.0 μL of an inosine-containing primer mixture, 2.0 μL of ethylene glycol, 0.5 μL of 25% (v/v) formamide and 1.5 μL of water for a total volume of 7.0 μL. This is allowed to sit at room temperature while the remaining reaction components are assembled.

The Enzyme Mix is prepared by mixing 0.378 μL of Enzyme Dilution Buffer, 0.381 μL of Bst DNA polymerase (120 units/μL), 0.2 μL SSB (5 μg/μL) and 0.041 μL of mutant Endonuclease V for a total volume of 1.0 μL.

A Reaction Mix is prepared by mixing 1.0 μL of 10× Reaction Buffer, 1.0 μL of Enzyme Mix and 1.0 μL of 50% (v/v) glycerol.

A complete Ping Pong reaction is prepared by mixing 7 μL of Denaturation Reaction with 3.0 μL of Reaction Mix and incubating at 45° for one hour. Note that other reaction components (e.g., Ficoll) may be substituted for water in the Denaturation Mix.

Following incubation the amplification is generally analyzed by gel electrophoresis using a 15% Acrylamide TBE-Urea gel (Invitrogen) Immediately prior to gel loading, 3 μL of a completed Ping Pong reaction is combined with 6 μL Gel Loading Buffer II (Invitrogen) and heat denatured at 95° C. for two minutes followed by immediate quenching on ice. 5 μL of this heat-denatured Ping Pong reaction is then loaded in one well of the gel. Electrophoresis is accomplished according to the manufacturer's (Invitrogen) instructions. Once electrophoresis is complete, the gel is stained with a 20× solution of SYBR Gold (Invitrogen) for 15 minutes and then scanned for fluorescein with a Typhoon™ 9410 Variable Mode Imager (GE Healthcare).

### Example 2

### Endonuclease-Assisted Isothermal Amplification of a Template DNA Using Exonuclease-Resistant, Inosine-Containing Primers

The primers used in endonuclease-assisted isothermal amplification contain an inosine as the penultimate 3′ base. Endonuclease V recognizes inosine as a non-natural base and nicks the DNA strand containing the inosine residue at one base 3′ to the lesion. An enhanced amplification kinetics may be obtained using a nuclease-resistant primer, wherein the phosphate bond between the inosine reside and the terminal 3′ base has been phosphorothioated.

Sequences for six forward and five reverse primers were identified in the 5′ region of the Mycobacterium tuberculosis rpoB gene. Two sets of these 11 primers were synthesized, one set without phosphorothioation and the other set with phosphorothioation between the inosine and the terminal 3′ base of each primer. TABLE 1 and TABLE 2 provide the sequences of the various primers used in the examples.

TABLE 1
Non-phosphorothioated primers without phosphorothioation between
the inosine and the terminal 3′ base of each primer

| Primer Name | Ref. No. | Sequence (5′→3′) | Length |
|---|---|---|---|
| IA TBropB F1 | SEQ ID NO: 7 | ACAGCCGCTAGTCCTAIT | 18 |
| IA TBropB F2 | SEQ ID NO: 8 | CCCGCAAAGTTCCTCIA | 17 |
| IA TBrpoB F3n | SEQ ID NO: 9 | ACCGGGTCTCCT TCIC | 16 |
| IA TBrpoB F4 | SEQ ID NO: 10 | GCTGCGCGAACCACTTIA | 18 |
| IA TBrpoB F5 | SEQ ID NO: 11 | CCGTACCCGGAGCIC | 15 |
| IA TBrpoB F6 | SEQ ID NO: 12 | CAGATTCCCGCCAGAIC | 17 |
| IA TBropB R2 | SEQ ID NO: 13 | GGCGAACCGATCAIC | 15 |
| IA TBropB R3 | SEQ ID NO: 14 | CGGCGGATTCGCIC | 14 |
| IA TBrpoB R4 | SEQ ID NO: 15 | GGTTGACATCACCCCIC | 17 |
| IA TBrpoB R5 | SEQ ID NO: 16 | GAGCACCTCTTCCAGIC | 17 |
| IA TBrpoB R6 | SEQ ID NO: 17 | CGATCGGAGACAGCTCIT | 18 |

TABLE 2
Phosphorothioated primers with phosphorothioation (* represents
phosphorothioate linkage) between the inosine and the terminal
3′ base of each primer.

| Primer Name | Ref. No. | Sequence (5′→3′) | Length |
|---|---|---|---|
| IA TBropB F1* | SEQ ID NO: 18 | ACAGCCGCTAGTCCTAI*T | 18 |
| IA TBropB F2* | SEQ ID NO: 19 | CCCGCAAAGTTCCTCI*A | 17 |
| IA TBrpoB F3n* | SEQ ID NO: 20 | ACCGGGTCTCCT TCI*C | 16 |
| IA TBrpoB F4* | SEQ ID NO: 21 | GCTGCGCGAACCACTTI*A | 18 |
| IA TBrpoB F5* | SEQ ID NO: 22 | CCGTACCCGGAGCI*C | 15 |
| IA TBrpoB F6* | SEQ ID NO: 23 | CAGATTCCCGCCAGAIC* | 17 |
| IA TBropB R2* | SEQ ID NO: 24 | GGCGAACCGATCAI*C | 15 |
| IA TBropB R3* | SEQ ID NO: 25 | CGGCGGATTCGCI*C | 14 |
| IA TBrpoB R4* | SEQ ID NO: 26 | GGTTGACATCACCCCI*C | 17 |
| IA TBrpoB R5* | SEQ ID NO: 27 | GAGCACCTCTTCCAGI*C | 17 |
| IA TBrpoB R6* | SEQ ID NO: 28 | CGATCGGAGACAGCTCI*T | 18 |

To generate the non-phosphorothioated primer set, 4.00 μL IA TBrpoB F1 (629 pmol/μL), 3.16 μL IA TBrpoB F2 (793 pmol/μL), 3.64 μL IA TBrpoB F3n (686 pmol/μL), 3.50 μL IA TBrpoB R2 (715 pmol/μL), 3.28 μl IA TBrpoB R3 (762 pmol/μL), 5.74 μL IA TBrpoB F4 (436 pmol/L), 2.84 μL IA TBrpoB F5 (880 pmol/μL), 4.17 μL IA TBrpoB F6 (599 pmol/μL), 4.94 μL IA TBrpoB R4 (506 pmol/μL), 4.15 μL IA TBrpoB R5 (602 pmol/μL) and 5.63 μL IA TBrpoB R6 (444 pmol/μL) was mixed with 204.95 μL HE(0.1)T buffer (Total Volume=250 μL)

To generate the phosphorothioated primer set, 3.32 μL IA TBrpoB F1*(754 pmol/μL), 2.72 μL IA TBrpoB F2* (920 pmol/μL), 2.83 μL IA TBrpoB F3* (883 pmol/μL), 4.68 μL IA TBrpoB F4* (534 pmol/μL), 1.72 μL IA TBrpoB F5* (1451 pmol/μL), 1.76 μL IA TBrpoB F6* (1417 pmol/μL), 2.72 μL IA TBrpoB R2* (920 pmol/μL), 1.80 μL IA TBrpoB R3* (1392 pmol/μL), 1.51 μL IA TBrpoB R4* (1652 pmol/μL), 1.79 μL IA TBrpoB R5* (1398 pmol/μl), and 7.00 μL IA TBrpoB R6* (358 pmol/μL) was mixed with 218.15 μL HE(0.1)T buffer (Total Volume=250 μL)

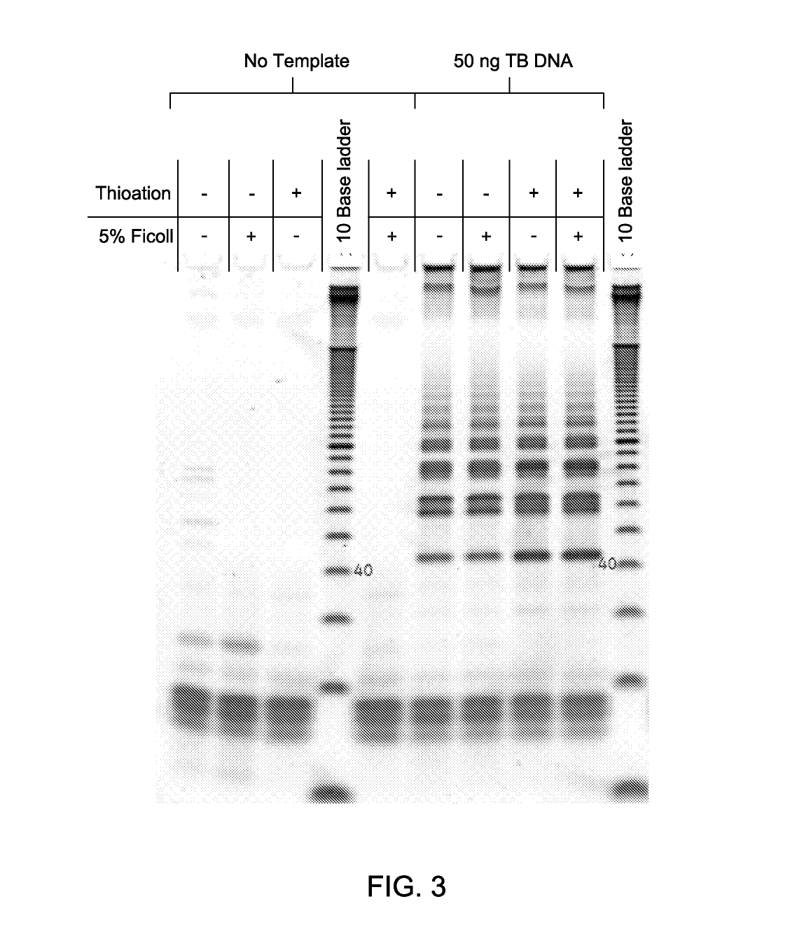

Each primer set was then used in an endonuclease-assisted isothermal amplification reaction prepared and analyzed as in Example 1. The final concentration of each oligonucleotide primer in the Ping Pong reaction was 10 pmol. As depicted in FIG. 3, use of nuclease-resistant primers (i.e., primer set containing phosphorothioation) increase the yield of endonuclease-assisted isothermal amplification reaction products by about a factor of two.

### Example 3

### Grafting of PEG Directly to Cellulosic Membrane Through Electron Beam Irradiation

Coating of and grafting to porous cellulose substrates (e.g., 903, 31ETF) or nitrocellulose (FF60, FF80HP) may be performed in an aqueous solution containing 10% PEGMA 300 (polyethylene glycol methyl ether methacrylate, Number average molecular weight (Mₙ) 300) and 30% Tween 20 followed by electron beam irradiation of the coated substrate. E-beam irradiation creates free radicals on the membrane surface which initiates polymerization of the methacrylate monomers, and as a result, the PEG moieties are permanently introduced to the membranes. After the e-beam treatment, the treated membranes are washed with water to remove co-solvent Tween 20 and ungrafted PEG species and then dried.

### Example 4

### Reduction of Non-Specific Binding of Proteins to the Pegylated Nitrocellulose Membranes

A running buffer was prepared by coating 40 nm gold nanoparticles with a final concentration of 0.2 mg/ml BSA and 0.8 OD (optical density) Au. Nitrocellulose (NC) membranes were dipped into the running buffer, and the gold label was used to serve as the indicator of BSA presence on the membranes. Cellulose absorbent pads were laminated on top of NC to ensure constant flow of running buffer on the NC membranes.

It was observed that for non-modified nitrocellulose, gold nanoparticles aggregated in the origin of the flow on the membrane, indicating non-specific binding of BSA to the membrane. Alternatively, it was observed that for PEG-modified nitrocellulose membranes, gold nanoparticle solutions were able to flow smoothly through the membrane into cellulose absorbent pad, indicating the intrinsic ability of the modified membrane to block non-specific interactions between the protein and the membrane.

### Example 5

### Endonuclease-Assisted Isothermal Amplification of a Template DNA on a Porous Matrix

Nine millimeter diameter discs of modified or unmodified porous matrices were sealed in hybridization chambers (e.g., Grace Bio-Labs SA8R-2.0-SecureSeal 8-9 mm Dia.×1.8 mm Depth, 26 mm×51 mm OD, 1.5 mm Dia. Ports) attached to new glass microscope slides. The porous matrix may be a FF60 nitrocellulose (GE Healthcare), PEGMA 300 grafted nitrocellulose (NC-PEG), 903 cellulose paper (GE Healthcare), PEGMA 300 grafted 903 cellulose paper (903-PEG), or a Fusion 5 paper (GE Healthcare). PEGMA 300 grafted nitrocellulose and PEGMA 300 grafted 903 cellulose paper were fabricated by soaking the appropriate base substrate (FF60 nitrocellulose or 903 cellulose) in an aqueous solution containing 10% (w/v) polyethylene glycol methyl ether methacrylate 300 (PEGMA 300; Sigma-Aldrich) and 30% (v/v) Tween 20 (Sigma-Aldrich). Excess solution was removed and the treated matrices were subjected to e-beam irradiation (Advanced Electron Beam (AEB) Application Development unit, EBLAB-150), with an operating voltage of 125 kV, and electron dosage delivery of 10 kGy. Following irradiation, the modified matrices were washed in distilled water by orbital rotating for 30 minutes, and repeat for three times. The membranes were then allowed to air dry at room temperature for overnight. The template DNA was 500 pg of TB genomic DNA. An amplification reaction was prepared and was added to each contained disc through a port. No denaturation of the double stranded DNA template was performed prior to amplification. The ports were sealed and the slide was incubated at 45° C. for one hour. Following incubation, the porous matrices were removed and placed in separate Costar® Spin-X® centrifuge tubes (Corning Life Sciences). The Spin-X tubes were centrifuged at 16×g for five minutes to collect the completed amplification reactions. Three microliters of each of the collected reaction were mixed with six microliters of Gel Loading Buffer II (Life Technologies) and denatured by heating at 95° C. for two minutes. The denaturations were quenched on ice and immediately loaded into separate wells of 15% acrylamide 7 M urea gels (Life Technologies). The gels were subjected to electrophoresis according to the manufacturer's instructions and then stained with SYBR Gold (Life Technologies). Stained gels were imaged using a Typhoon™ Variable Mode Imager (GE Healthcare).

### Example 6

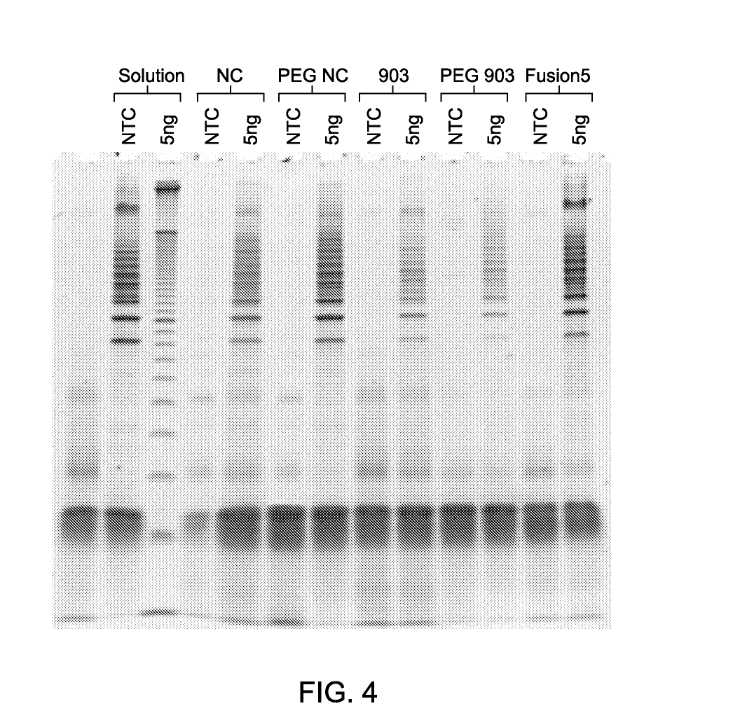

Isothermal amplification reactions were prepared and processed according to Example 2 using an exonuclease-resistant primer set designed to generate an amplicon of 81 base pairs in length from a 5′ region of the Mycobacterium tuberculosis rpoB gene. Porous matrices of FF60 nitrocellulose (GE Healthcare), PEGMA 300 grafted nitrocellulose, 903 cellulose (GE Healthcare), PEGMA 300 grafted 903 cellulose and Fusion 5 (GE Healthcare) were tested for their ability to support amplification as outlined in Example 5. Five nanograms of purified M. tuberculosis double stranded DNA (American Type Culture Collection) were used as the template in each reaction. FIG. 4 demonstrates that every porous matrix tested supported amplification to some degree as compared to the reaction completed in solution.

### Example 7

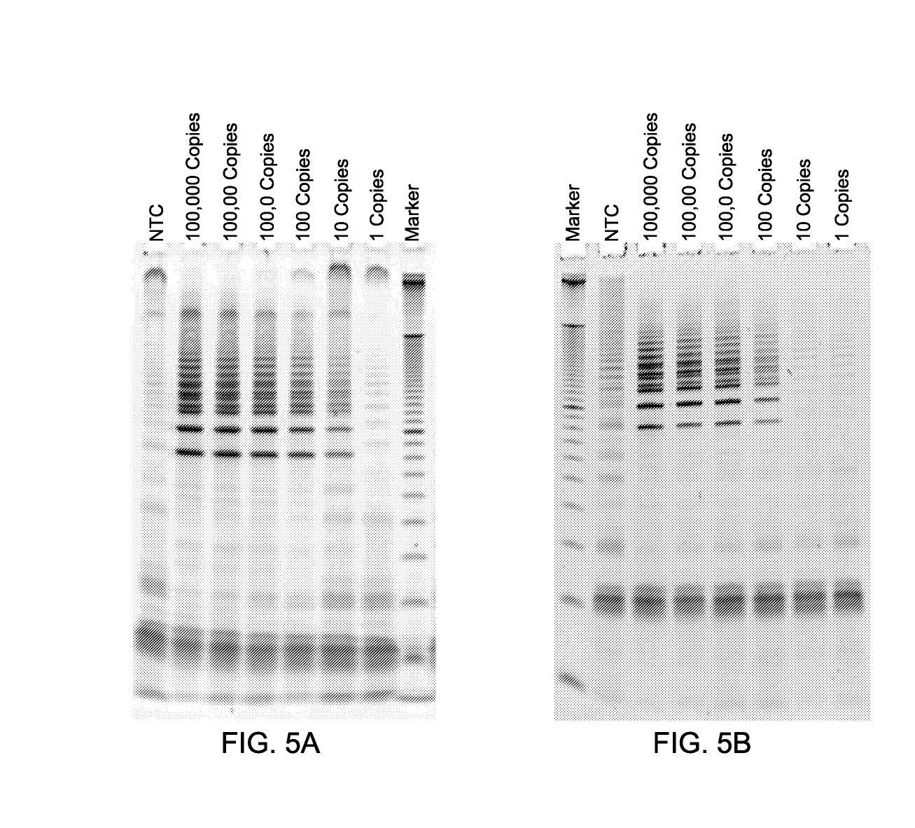

A titration of input template amount of M. tuberculosis was accomplished to compare in solution amplification to in paper amplification. One genome equivalent of M. tuberculosis DNA was estimated to be 4,403,765 base pairs in length or 4.78 fg. The primer set used is provided in Table 3. In FIG. 5A, in solution amplification detected down to 10 copies of input template, with nonspecific amplification products appearing only in the one copy reaction. In FIG. 5B, in paper amplification produced the expected amplification products in all reactions, demonstrating effective amplification of double stranded target DNA in a porous matrix.

Primer Set DNA Sequences (I designates an inosine moiety; * designates phosphorothioation)

TABLE 3
Phosphorothioated primers with phosphorothioation
(* represents phosphorothioate linkage) between the
inosine and the terminal 3' base of each primer.

| | | | Amount |
|---|---|---|---|
| | | | in one |
| | | | reaction |
| Name | Sequence | Length | (pmol) |
| SEQ ID NO: 29 | CATGAAGTGCTGGAAGGATI*C | 21 | 4 |
| SEQ ID NO: 30 | TCCTCTAAGGGCTCTCGTTI*G | 21 | 4 |
| SEQ ID NO: 31 | AAATTATCGCGGCGAACGGI*C | 21 | 6 |
| SEQ ID NO: 32 | GGCAGATTCCCGCCAGAI*C | 19 | 6 |
| SEQ ID NO: 33 | AAAACAGCCGCTAGTCCTAI*T | 21 | 8 |
| SEQ ID NO: 34 | TCGCCCGCAAAGTTCCTCI*A | 20 | 8 |
| SEQ ID NO: 35 | CCAAACCGGGTCTCCTTCI*C | 20 | 10 |
| SEQ ID NO: 36 | TAAGCTGCGCGAACCACTTI*A | 21 | 10 |
| SEQ ID NO: 37 | CTGGGTTGACATCACCCCI*C | 20 | 10 |
| SEQ ID NO: 38 | AAGTCCTCGATCGGAGACAI*C | 21 | 10 |
| SEQ ID NO: 39 | GACAACGACATCGACCCGI*A | 20 | 8 |
| SEQ ID NO: 40 | CGTCGAAACGAGGGTCAGAI*A | 21 | 8 |
| SEQ ID NO: 41 | CTCGTCGACGGGTGCCTTI*A | 20 | 8 |
| SEQ ID NO: 42 | GTACGTCATGTCCTTGTCTTTI*C | 23 | 6 |
| SEQ ID NO: 43 | TCACCGGTGTTGTTGTTGATI* A | 22 | 4 |

### Example 8

### Effect of Blocking Agents and/or Other Additives on Endonuclease-Assisted Isothermal Amplification of Target dsDNA in Solution and in Different Porous Matrices

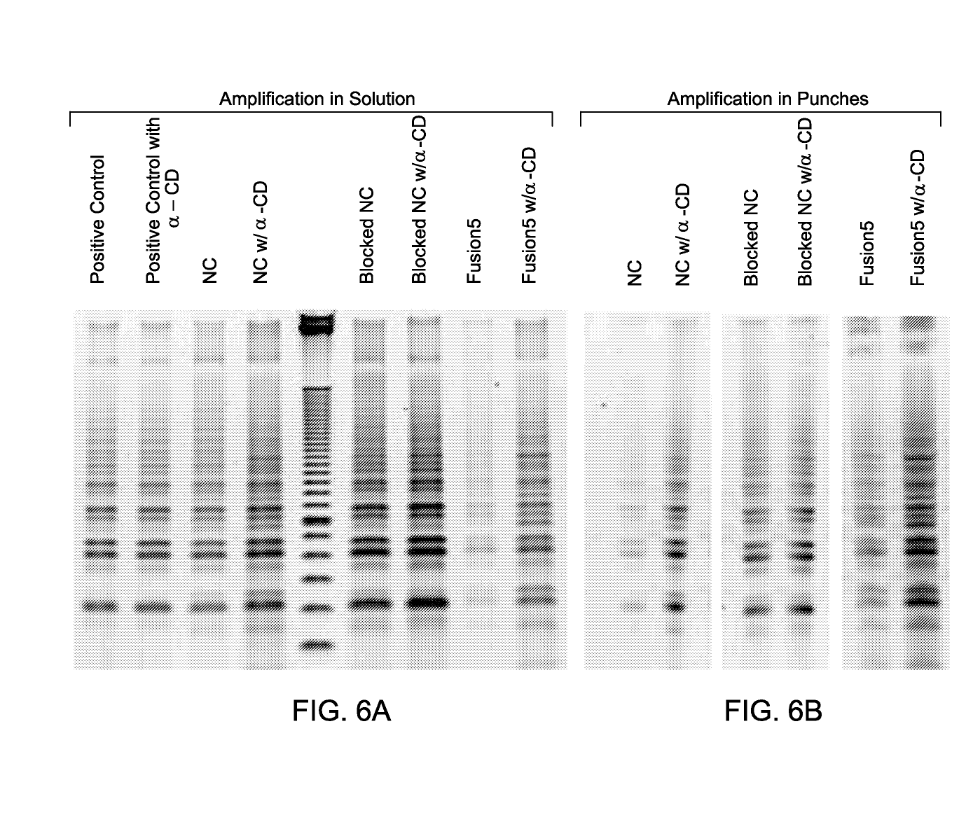

Ping pong reactions were carried out using the modified primers. Reactions took place within the porous matrix. Following incubation, the porous matrices were removed and placed in separate Costar® Spin-X® centrifuge tubes (Corning Life Sciences). The Spin-X tubes were centrifuged at 16×g for five minutes to collect the completed amplification reactions. Three microliters of each reaction (FIG. 6A) of the collected reaction were mixed with six microliters of Gel Loading Buffer II (Life Technologies) and denatured by heating at 95° C. for two minutes. The denaturations were quenched on ice and immediately loaded into separate wells of 15% acrylamide 7 M urea gels (Life Technologies). A second gel was made by inserting the porous matrix directly in to the gel (FIG. 6B), and adding Gel Loading Buffer II (Like Technologies). The label “pos.control” in FIGS. 6A and 6B refers to positive control. The gels were subjected to electrophoresis according to the manufacturer's instructions and then stained with SYBR Gold (Life Technologies). Stained gels were imaged using a Typhoon™ Variable Mode Imager (GE Healthcare).

FIG. 6A illustrates the effects blocking agents and/or other additives on endonuclease-assisted isothermal amplification of a target dsDNA in solution and FIG. 6B illustrates the effects blocking agents and/or other additives on endonuclease-assisted isothermal amplification of a target dsDNA in different porous matrices. In absence of blocking agent, the non-modified matrixes showed minimal or mediocre DNA amplification on different porous matrices. However, the modified porous matrices (e.g., PEGylated Nitrocellulose (NC-PEG)) shows enhanced DNA amplification efficiencies. Alpha-cyclodextrin enhanced the DNA amplification in many porous matrices.

The above detailed description is exemplary and not intended to limit the invention of the application and uses of the invention. Throughout the specification, exemplification of specific terms should be considered as non-limiting examples. The singular forms “a”, “an” and “the” include plural referents unless the context clearly dictates otherwise. Approximating language, as used herein throughout the specification and claims, may be applied to modify any quantitative representation that could permissibly vary without resulting in a change in the basic function to which it is related. Accordingly, a value modified by a term such as “about” is not to be limited to the precise value specified. Unless otherwise indicated, all numbers expressing quantities of ingredients, properties such as molecular weight, reaction conditions, so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the invention. At the very least, and not as an attempt to limit the application of the doctrine of equivalents to the scope of the claims, each numerical parameter should at least be construed in light of the number of reported significant digits and by applying ordinary rounding techniques. Where necessary, ranges have been supplied, and those ranges are inclusive of all sub-ranges there between.

The invention may be embodied in other specific forms without departing from the spirit or essential characteristics thereof. The foregoing embodiments are selected embodiments or examples from a manifold of all possible embodiments or examples. The foregoing embodiments are therefore to be considered in all respects as illustrative rather than limiting on the invention. While only certain features of the invention have been illustrated and described herein, it is to be understood that one skilled in the art, given the benefit of this disclosure, will be able to identify, select, optimize or modify suitable conditions/parameters for using the methods in accordance with the principles of the invention, suitable for these and other types of applications. The precise use, choice of reagents, choice of variables such as concentration, volume, incubation time, incubation temperature, and the like may depend in large part on the particular application for which it is intended. It is, therefore, to be understood that the appended claims are intended to cover all modifications and changes that fall within the spirit of the invention. Further, all changes that come within the meaning and range of equivalency of the claims are intended to be embraced therein.

1. A method of producing at least one amplicon based on a target double stranded DNA within a porous matrix comprising:
(a) providing the porous matrix;
(b) impregnating the target double stranded DNA within the porous matrix;
(c) contacting the impregnated, target double stranded DNA with a DNA amplification reaction mixture comprising at least one inosine-containing primer, at least one 5′→3′ exonuclease-deficient DNA polymerase having strand displacement activity, at least one nuclease that is capable of nicking a DNA at a residue 3′ to an inosine residue, and a dNTP mixture;
(d) amplifying at least one portion of the impregnated target double stranded DNA within the porous matrix using the DNA amplification reaction mixture of step (c) to produce the at least one amplicon within the porous matrix; and
(e) determining a rate of production of the at least one amplicon within the porous matrix.

2. The method of claim 1, further comprising determining a presence, an absence, or a quantity of the amplicon within the porous matrix.

3. The method of claim 1, wherein the at least one portion of the impregnated target double stranded DNA is amplified within the porous matrix under isothermal conditions without denaturing the target double stranded DNA.

4. The method claim 1, wherein the porous matrix comprises a cellulose membrane, a nitrocellulose membrane, a cellulose acetate membrane, a nitrocellulose mixed ester membrane, a glass fiber, a polyethersulfone membrane, a nylon membrane, a polyolefin membrane, a polyester membrane, a polycarbonate membrane, a polypropylene membrane, a polyvinylidene difluoride membrane, a polyethylene membrane, a polystyrene membrane, a polyurethane membrane, a polyphenylene oxide membrane, a poly(tetrafluoroethylene-co-hexafluoropropylene) membrane, or a combination thereof.

5. The method of claim 4, wherein the porous matrix is a cellulose membrane, a nitrocellulose membrane, or a combination thereof.

6. The method of claim 4, wherein the porous matrix further comprises an alkyl oligo(oxyalkylene) group.

7. The method of claim 6, wherein the porous matrix is a polyethyleneglycol-modified cellulose membrane, polyethyleneglycol-modified nitrocellulose membrane, or a combination thereof.

8. The method claim 1, wherein the porous matrix comprises a detergent.

9. The method of claim 8, wherein the amplification mixture comprises a detergent sequestering agent.

10. The method of claim 9, wherein the detergent sequestering agent is an alpha-cyclodextrin.

11. The method of claim 1, wherein the nuclease is an endonuclease that is capable of nicking an inosine-containing strand of a double stranded DNA at a residue 3′ to an inosine residue.

12. The method of claim 11, wherein the endonuclease is endonuclease V or a mutant endonuclease V.

13. The method of claim 12, wherein the mutant endonuclease V is chosen from a mutant Escherichia coli endonuclease V comprising amino acid sequence of SEQ ID NO: 2, a mutant Archaeoglobus fulgidus endonuclease V comprising amino acid sequence of SEQ ID NO: 4, or a mutant Termotoga maritima endonuclease V comprising amino acid sequence of SEQ ID NO: 6.

14. The method of claim 1, wherein the at least one 5′→3′ exonuclease-deficient DNA polymerase is selected from 5′→3′ exonuclease-deficient T7 DNA polymerase, 5′→3′ exonuclease-deficient Bst DNA polymerase, 5′→3′ exonuclease-deficient Klenow, 5′→3′ exonuclease-deficient delta Tts DNA polymerase, or combinations thereof.

15. The method of claim 1, wherein the target double stranded DNA is a genomic DNA.

16. The method of claim 1, wherein the DNA amplification reaction mixture further comprises a detergent, a blocking agent, or both.

17. The method of claim 1 or claim 6, wherein the DNA amplification mixture further comprises gelatin, powdered milk, albumin, casein, bactopeptone or combinations thereof.

18. The method of claim 1, further comprising eluting out the amplicon from the porous matrix.

19. The method of claim 1, wherein the inosine-containing primer is an exonuclease-resistant primer.

20. A method for extracting a genetic material from a biological sample comprising:
(a) contacting the biological sample with a porous matrix comprising chemicals that lyse the biological sample and preserve the genomic DNA within the porous matrix;
(b) contacting the preserved genomic DNA within the porous matrix with a DNA amplification reaction mixture comprising at least one inosine-containing primer, at least one 5′→3′ exonuclease-deficient DNA polymerase having strand displacement activity, at least one nuclease that is capable of nicking a DNA at a residue 3′ to an inosine residue, and dNTP mixture;
(c) amplifying at least one portion of the preserved genomic DNA within the porous matrix using the DNA amplification reaction mixture of step (b) to produce t at least one amplicon within the porous matrix;
(d) determining a quantity of amplicons produced within the porous matrix; and
(e) eluting the at least one amplicon out of the porous matrix.

21. The method of claim 20, wherein the porous matrix is impregnated with chemicals chosen from a salt, a detergent, a chaotrope, a reducing agent, an anti-oxidant, a chelating agent, a buffer, or combinations thereof.

22. The method of claim 21, wherein the porous matrix is impregnated with guanidinium hydrochloride, arginine, sodium dodecyl sulfate (SDS), urea, or combinations thereof.

23. The method of claim 22, wherein the endonuclease is endonuclease V or a mutant endonuclease V.

24. The method of claim 23, wherein the DNA amplification mixture further comprises gelatin, powdered milk, albumin, casein, bactopeptone or combinations thereof.
